(19) United States
(12) Patent Application Publication
(10) Pub. No.: US 2025/0107779 A1
(43) Pub. Date: Apr. 3, 2025

(54) TIME-SHARED TRANSMISSION AND CONTINUOUS RECORDING OF ULTRASOUND DATA FOR ENHANCED FRAME RATES

(71) Applicant: CLOUDSTREAM MEDICAL IMAGING, INC., Houston, TX (US)

(72) Inventors: Chengbin PENG, Houston, TX (US); Jun TANG, Houston, TX (US)

(21) Appl. No.: 18/832,077

(22) PCT Filed: Jan. 30, 2023
(86) PCT No.: PCT/US2023/011823

§ 371 Date: Jul. 22, 2024

Related U.S. Application Data
(60) Provisional application No. 63/304,633, filed on Jan. 30, 2022.

(51) Int. Cl. A61B 8/00; A61B 8/08; G01S 7/52; G01S 15/89

(52) U.S. Cl. CPC A61B 8/5207; A61B 8/08; G01S 7/5209; G01S 15/89

(57) ABSTRACT

A method of acquiring ultrasound radio-frequency data includes providing an ultrasound transducer; providing an ultrasound data acquisition system; transmitting ultrasound beams using a transmission function of the ultrasound transducer and ultrasound data acquisition system; receiving the ultrasound beams using a reception function of the ultrasound transducer and the ultrasound data acquisition system; recording raw radio-frequency data with the ultrasound data acquisition system; sending the raw radio-frequency data to a processing unit; and deblending the raw radio-frequency data into individual ultrasound beam records. The ultrasound beams are transmitted in such a way that a subsequent ultrasound beam is transmitted before a previous ultrasound beam is received by the ultrasound transducer, and the ultrasound beams are overlapping in time in accordance with a first pseudo random sequence and overlapping in space in accordance with a second pseudo random sequence. A system for acquiring and processing BLEND ultrasound radio-frequency data is also disclosed.

This application claims priority to U.S. Provisional Patent Application No. 63/304,633, filed on Jan. 30, 2022, which is incorporated by reference for all purposes as if fully set forth herein.

### FIELD OF THE INVENTION

The present invention relates to the acquisition and processing of ultrasound data for medical applications. In particular, the invention addresses two competing needs in medical diagnostic imaging: (1) high frame rate for imaging moving tissues and organs, such as blood and heart, (2) accurate detection of small lesions that requires sharp resolution and high signal to noise ratio.

### BACKGROUND OF THE INVENTION

Medical ultrasound imaging for diagnosis has advantages, such as reasonable cost, real-time imaging, portability, and its harmless effect, over computerized tomography (CT) and magnetic resonance imaging (MRI) [1, 2]. However, the resolution of the ultrasound imaging system is usually lower than that of CT and MRI systems [3]. Ultrasound imaging technology is progressing towards high quality and sharp resolution, thanks to better data acquisition hardware and sophisticated processing software [4].

Commonly used ultrasound data acquisition methods for medical applications include focused beams and planewave beams. In ultrasound data acquisition using focused beams the time delay of each transmitter is electronically controlled in such a way that transmitters employed by this beam emit waves that arrive at a focal point at the same time. The in-sonification at the focal point is very strong and it rapidly dies down away from the focal point. In ultrasound data acquisition using planewave beams the time advance of each transmitter is similar to that of a divergent beam except the virtual focal point is far away behind the transducer. All transmitters participate in the excitation of each planewave beam. The in-sonification of a planewave beam in the image domain is weak and uniform. Most commercial ultrasound scanners employ a focused beam data acquisition because the signal to noise ratio is much higher thanks to the focusing ability of focused beams.

Ultrasound beams are acquired sequentially, one after another, in most traditional data acquisition methods. The time required to collect each beam is dictated by the travel time of echoes from the transducer to deepest tissues under examination. The total lapse time for collecting a complete dataset for producing one frame of image is the product of the number of beams and the time required for one beam. The temporal resolution, expressed in number of frames per second (NFS), is the inverse of the total lapse time. A typical value for the number of beams required for a high-quality image is on the order of 200. A typical value for data recording time for one beam is on the order of 200 microseconds. The frame rate is approximately 25 for data acquisition using focused beams. For planewave data acquisition, one beam still takes about 200 microseconds to collect. If we only use one planewave beam in beamforming then the frame rate can be as high as 5000 [5]. If we use 100 planewave beams in beamforming then the frame rate will drop to 50. The higher frame rate using planewave beams is achieved at the expense of both resolution and signal to noise ratio in the final image. The same can be said of other suggestions that use a few focused beams to achieve higher frame rate [6].

### SUMMARY OF THE INVENTION

In one embodiment, the present application discloses a method of acquiring ultrasound radio-frequency data using a time-shared transmission and continuous recording (BLEND) design. The method includes: providing an ultrasound transducer, the ultrasound transducer including a plurality of elements; providing an ultrasound data acquisition system, the ultrasound data acquisition system including analog electronics, an analog-to-digital converter, and a CPU (central processing unit) or GPU (graphic processing unit); transmitting a plurality of ultrasound beams using a transmission function of the ultrasound transducer and the ultrasound data acquisition system; receiving the ultrasound beams using a reception function of the ultrasound transducer and the ultrasound data acquisition system; recording raw radio-frequency data with the ultrasound data acquisition system; sending the raw radio-frequency data to a processing unit; and deblending the raw radio-frequency data into individual ultrasound beam records. The ultrasound beams are transmitted in such a way that a subsequent ultrasound beam is transmitted before a previous ultrasound beam is received by the ultrasound transducer; and the ultrasound beams are overlapping in time in accordance with a first pseudo random sequence and overlapping in space in accordance with a second pseudo random sequence.

In another embodiment, the ultrasound transducer is a 1D linear array, 1D curved array, 1D phased array, or 2D matrix array.

In another embodiment, the elements are first used as emitters and subsequently used as receivers.

In another embodiment, the elements include first row elements and second row elements; the first row elements are dedicated to transmission or reception; and the second row elements are dedicated to reception or transmission.

In another embodiment, the elements include odd elements and even elements; the odd elements are dedicated to transmission or reception; and the even elements are dedicated to reception or transmission.

In another embodiment, the data acquisition system includes analog electronics, one or more analog-to-digital converters, and one or more CPUs (or GPUs).

In another embodiment, a time gap (dither time) between two adjacent ultrasound beams is randomly chosen between 0 and 200 microseconds.

In another embodiment, deblending the ultrasound radio-frequency data includes: (i) taking the ultrasound radio-frequency data as an input; (ii) extracting raw beam records by reversing dither times and applying receiver apodizations for the ultrasound beams; (iii) sorting the raw beam records into a common receiver domain; (iv) performing de-spike and random noise attenuation in the common receiver domain; and (v) resorting the processed data back to obtain the individual ultrasound beam data.

In another embodiment, the present application provides a system for acquiring and processing ultrasound radio-frequency (RF) data using a time-shared transmission and continuous recording (BLEND) design. The system includes: an ultrasound transducer, the ultrasound transducer including a plurality of elements; a data acquisition system, the data acquisition systems including analog electronics, an analog-to-digital converter, and a first CPU (central processing unit) or first GPU (graphic processing unit); a display device; a keyboard; a pointing device; a data acquisition device that includes analog to digital converters (ADC); and a processing unit that includes a second CPUs or a second GPUs. The first and second CPUs and the first and second GPUs are adapted to: acquire, via the ultrasound transducer and the data acquisition system, raw radio-frequency data by transmitting a plurality of ultrasound beams in such a way that a subsequent ultrasound beam is transmitted before a previous ultrasound beam is received by the ultrasound transducer; record the raw radio-frequency data with the data acquisition system; send the raw radio-frequency data to the processing unit; deblend the raw radio-frequency data into individual ultrasound beam data; process and send the deblended ultrasound beam data to CPU memories or GPU memories; beamform the deblended ultrasound beam data on the first and second CPUs or the first and second GPUs to obtain an ultrasound image; and process and send the ultrasound image to the display device.

In another embodiment, the display device is connected to the processing unit remotely, via internet connection, wireless connection, or satellite connection.

In another embodiment, the ultrasound transducer is a 1D linear array, 1D curved array, 1D phased array, or 2D matrix array.

In another embodiment, the keyboard is a wireless keyboard or a software keyboard installed on the processing unit.

In another embodiment, the elements are first used as emitters and subsequently used as receivers.

In another embodiment, the elements include first row elements and second row elements; the first row elements are dedicated to transmission or reception; and the second row elements are dedicated to reception or transmission.

In another embodiment, the elements include odd elements and even elements; the odd elements are dedicated to transmission or reception; and the even elements are dedicated to reception or transmission.

In another embodiment, deblending the raw radio-frequency data includes: (i) taking the ultrasound radio-frequency data as an input; (ii) extracting raw beam records by reversing dither times and applying receiver apodizations for the ultrasound beams; (iii) sorting the raw beam records into a common receiver domain; (iv) performing de-spike and random noise attenuation in the common receiver domain; and (v) resorting the processed data back to obtain the individual ultrasound beam data.

In another embodiment, the data acquisition system and the processing unit share a same CPU or a same GPU, and the first CPU is the same as the second CPU or the first GPU is the same as the second GPU.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 is a sketch of a BLEND record for a transducer with 128 elements. The vertical axis is element position. The horizontal axis is lapse time which is long (on the order of 100,000 samples in time). Different beams are recorded at random dither times and in a random order. In this illustration, B15 stands for beam #15, B72 is beam #72, and so on.

FIG. 2 is an illustration of a transmission design of BLEND ultrasound data acquisition using a 128-element linear array transducer placed vertically. T0 is the time recording function is activated. t0 is the dither time of a given beam. The Tx delay is the difference between time stamp at the probe center and time stamp of a transmitter.

FIG. 3 is an illustration of a reception design of BLEND ultrasound data acquisition using a 128-element linear array transducer placed vertically. The horizontal axis is lapse time. The recording circuitry is always on, continuously recording signals arriving at the analog to digital converters (ADC). Each element is switched on and off many times for transmission and reception: off when the element is used for transmission and on after the transmission is completed. In this illustration white area is when reception is on (>95%), and black area is when reception is off (<5%).

FIG. 4 shows a phantom model for a simulation: white dots are point scatters and white lines are reflectors in the phantom model.

FIG. 5 shows comparisons of raw data of a focused beam in conventional acquisition (left) and an extracted record of the same beam in blended acquisition (right). The BLEND record contains interferences from many neighboring beams.

FIG. 6 shows (a) a BLEND beam record, (b) a common receiver gather of the BLEND data, and (c) the same beam record after deblending.

FIG. 7 shows the comparisons of a focused beam image with conventional data acquisition (left) and the same image of blended data with BLEND data acquisition (right): The same focused beams is used in both images. The data collection time in the conventional data acquisition (left) is 38.4 milliseconds and the data collection time in BLEND data acquisition (right) is 5.0 milliseconds. All displays are shown in 60 dB.

FIG. 8 is a schematic representation of the BLEND data acquisition and imaging architecture of one embodiment of the present invention.

### DETAILED DESCRIPTION OF THE ILLUSTRATED EMBODIMENTS

Reference will now be made in detail to embodiments of the present invention, example of which is illustrated in the accompanying drawings.

The present invention proposes a novel design for acquiring radio-frequency ultrasound data (also called RF data) using a linear 1D array, a curved 1D array, a phased 1D array, or a matrix 2D array transducer. In this design the recording circuitry of a data acquisition system is always turned on, continuously recording signals arriving at the analog to digital converters (ADC). At certain time intervals short pulse excitations are transmitted into tissues under examination. The transmitters used for a beam are immediately switched into receiving mode upon completion of their firings unless they are dedicated to transmission only. The process is repeated until all required beams are transmitted and received. The beams are dithered in time in a pseudo random sequence. They are also separated in space in another pseudo random sequence. We call this data acquisition design and process time-shared transmission and continuous recording, or BLEND for short. The BLEND data acquisition method is unique and efficient, achieving a much higher frame rate without appreciable loss of image quality. Proper deblending of a BLEND record are required prior to beamforming. After deblending the RF data are fed into a beamformer that is specific for each beam type. Good image quality and very high frame rate are both achieved with our BLEND design and process.

We call our invention time-shared transmission and continuous recording, or BLEND for short. It is a new way of acquiring raw ultrasound data. We permit time share in both transmission and reception of ultrasound beams. The time share is designed in such a way that would permit us to de-blend the measured data to recover individual ultrasound beams. Our BLEND design and process is the fastest method to acquire the same amount of ultrasound beam data. The ability to rapidly illuminate a large volume of tissues with in-sonification of many ultrasound beams at overlapping time makes our BLEND design and process unique and suitable for diagnosing cardiovascular diseases, heart diseases, blood blockages, and micro blood flows around malignant cancers, all demanding both high frame rate and high image quality.

### Technical Description

Focused ultrasound beams are widely used in commercial diagnostic imaging of tissues and organs because of its high resolution and high signal to noise ratio [1, 3]. Less common are divergent ultrasound beams and planewave ultrasound beams. High frame rates in data acquisition are necessary for imaging objects in motion, such as blood flows, hearts, and micro vibrations of tissues. Ultrafast imaging can be achieved using one or a few planewave beams at the expense of image resolution and signal to noise ratio [5]. We propose a new data acquisition method that can achieve both high frame rates and good image quality (both resolution and signal to noise ratio) at the same time. We call our special design and process time-shared transmission and continuous recording (BLEND).

### Part I: Time-Shared Transmission and Continuous Recording (BLEND)

### 1.1 Definition of BLEND

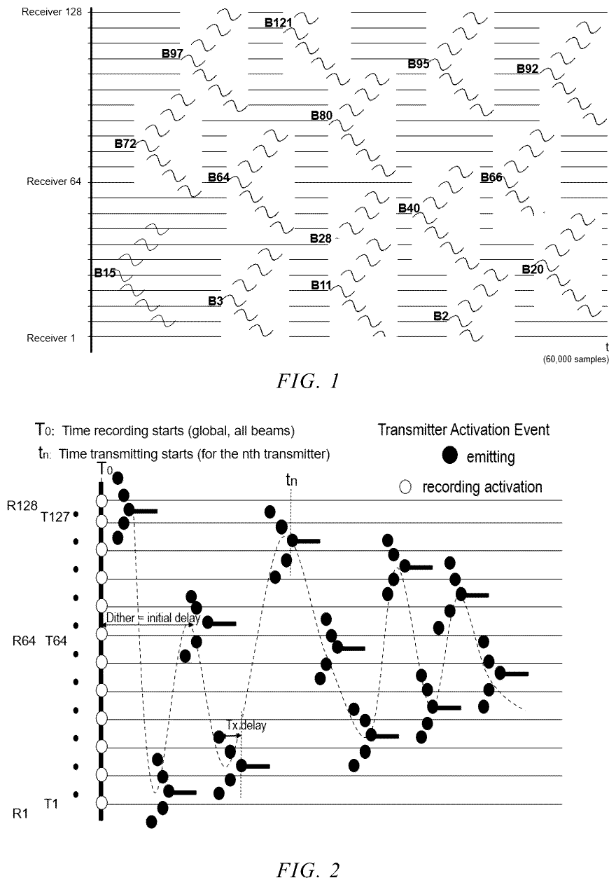

In our time-shared transmission and continuous recording (BLEND) design the recording circuitry is turned on all the time during acquisition of a frame-worth ultrasound data. A subsequent beam is transmitted before the previous beams are completely recorded. The spatial sequence of beams is random and separated. The time gap (also called dither time) between two adjacent beams is also randomly chosen. This special design and process are aimed at shortening data acquisition time and reducing data size. All beams are blended and stored into one long data buffer, hence the nickname BLEND. There is no clear separation of one beam from other beams in the blended data (FIG. 1).

As an illustrative example in FIG. 1, we choose a linear array transducer with 128 elements. Let's assume we need 256 focused beams for one frame-worth ultrasound data, each beam has 3000 samples in time (or 150 microseconds, assuming a data sampling rate of 20 MHz). For conventional data acquisition the total data size will be 256×128×3000×4=393.2 MB, and the total data collection time will be: 150×256=38,400 microseconds. The maximum frame rate is less than 26. For our BLEND data acquisition, we use a dither time of 20 microseconds, which will allow recording of 6 overlapping beams. The record buffer has 10,000 samples in time and each has 4 bytes. The data size will be 128 (channels)×100,000 (data samples)×4 (bytes)=51.2 MB. And the total data collection time will be: 100,000 (data samples)×0.05 (sampling rate in microseconds)=5,000 microseconds. The maximum frame rate is now close to 200. We achieve, for the same number of ultrasound beams, 86.9% reduction in data size, 86.9% reduction in data acquisition time, and approximately 600% increase in frame rate.

### 1.2 BLEND Transmission Design

FIG. 2 shows the transmission design for acquisition of BLEND ultrasound data. The horizontal axis is lapsed time and the vertical axis is element position. We use a linear array of 128 elements as an example. The beam type is focused beam. The design equally applies to other array configurations, such as linear arrays with more than or less than 128 elements, curved arrays with arbitrary number of elements, or phased arrays with arbitrary number of elements. The design also equally applies to all beam types: focused beam, divergent beam, planewave beam, and other beam types. In FIG. 2 the transducer is placed vertically at the left. Acoustic waves propagate from left to right into human tissues. A set of focused beams are transmitted at different times (also called dither times). Each beam has its own settings for Tx aperture and Rx aperture. The dither times are spaced randomly between (T0, Tmax), approximately at 20-40 microseconds interval. The firing sequence of beams is also randomized. In this way we can achieve better deblending results in subsequent data processing steps prior to beamforming.

### 1.3 BLEND Reception Design

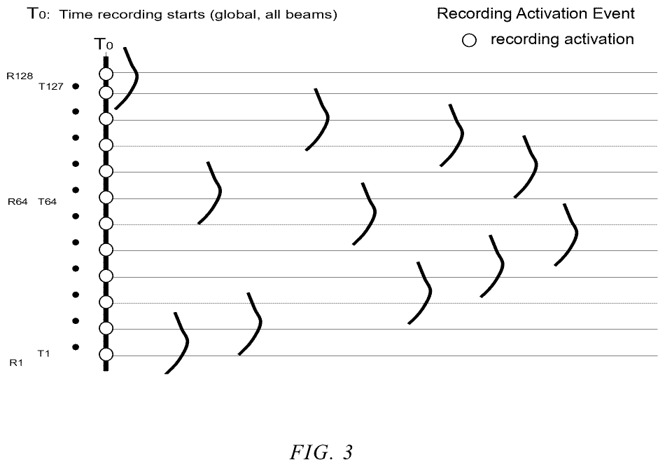

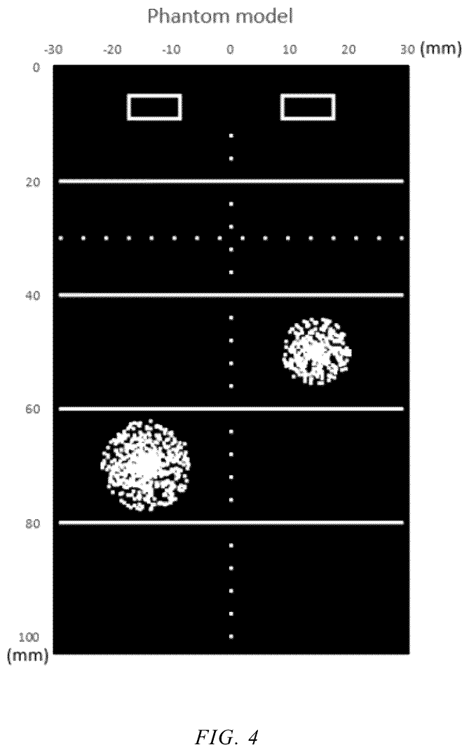

FIG. 3 shows the reception design of BLEND ultrasound beam acquisition. The horizontal axis is lapsing time and the vertical axis is element position. We use a linear array of 128 elements as an example. Other array configurations work equally well. The recording circuitry is turned on at TO and remains on all the time, continuously recording signals arriving at the ADC. Each receiving element on the transducer may be turned on and off many times: off when the element is used for transmission and on after the transmission event is completed. The process is repeated until all beams are transmitted. The thick black lines are time intervals when elements are used for transmission. No useful data is recorded in the time intervals marked by the black lines. These black lines are data gaps. These gaps are small and random, having very little impact on image quality.

### 1.4 Other Design Considerations

There are several variations in the design of BLEND. One design uses the same row of elements on a transducer for transmission and reception in which an element is first used as an emitter and subsequently used as a receiver. Another design calls for two rows of elements on a transducer, one row is dedicated to transmission and the other row is dedicated to reception. Yet another design alternates elements on a transducer: odd elements for transmission and even elements for reception, or vice versa. In all designs the recording circuitry is continuously recording signals nonstop. In the second and third designs there are no data gaps because there is no time sharing between transmitting and receiving for a given element.

### Part II: Wavefield Separation of a BLEND Record

A BLEND record contains all ultrasound beams required for producing a high-quality image. These beams are overlapping in time and space in accordance with two pseudo random sequences: one for dither time and another one for spatial location of each beam. In this section we disclose a method for deblending a BLEND record into individual ultrasound beam data.

### 2.1 Separation of a BLEND Record into Beams

An input data sample in a BLEND record at receiver location xⱼ and at time t(B(xⱼ, t)) is the superposition of all beams with time dithers and apodizations:

$$B ( {x}_{j} , t ) = {\sum }_{i = 1}^{{N}_{b}} {α}_{ij} {S}_{i} ( {x}_{j} , t - {δ}_{i} ) \qquad ( 1 )$$

where N_{b} is the total number of beams, Sᵢ(xⱼ, t) represents the data of i-th beam at the receiver location, δᵢ is the dither time of the i-th beam, αᵢⱼ is the receiver apodization of the j-th receiver for the i-th beam. The set of dither time {δᵢ} is a pseudo random sequence. The firing order of beams is in another pseudo random sequence.

The individual beam data in equation (1) is another superposition of acoustic responses of individual transmitters with a set of transmitter delays (also called Tx delays) and apodizations:

$${S}_{i} ( {x}_{j} , t ) = {\sum }_{k = 1}^{M} {α}_{ik} u ( {x}_{k} , {x}_{j} , t + {τ}_{ik} ) \qquad ( 2 )$$

where M is the total number of elements on a transducer, u(xₖ xⱼ, t) represents the individual acoustic response of a transmitter at xₖ and receiver at xⱼ, τᵢₖ is the Tx delay of the k-th transmitter for the i-th beam, αᵢₖ is the transmitter apodization of the k-th transmitter for the i-th beam. We typically set αᵢⱼ or αᵢₖ to 1 if the transmitter or the receiver is active, otherwise we set them to zero.

Combining equation (1) and (2) together we get an expression for the BLEND record in terms of acoustic responses of individual transmitters (also known as synthetic aperture radar records):

$$B ( {x}_{j} , t ) = {\sum }_{i = 1}^{{N}_{b}} {α}_{ij} {\sum }_{k = 1}^{M} {α}_{ik} u ( {x}_{k} , {x}_{j} , t + {τ}_{ik} - {δ}_{i} ) \qquad ( 3 )$$

Equation (3) is accurate for all beam types: focused beam, divergent beam, planewave beam, to name a few. Their differences are in the Tx-delay setting. Equation (3) is also accurate for all sub-apertures as the apodizations of both transmitters and receivers can be arbitrarily set. The formulation in equation (3) applies to 2D and 3D cases equally well, as the coordinates xₖ and xⱼ can be vectors in (x, y) plane.

The key to separation of a BLEND record into beams (per equation (1)) or synthetic aperture radar record (per equation (3)) is to take advantage of pseudo randomness of both the dither time and the beam firing sequence. The extraction of a beam from a BLEND record can be simply written as:

$${S}_{i} ( {x}_{j} , t ) = {α}_{ij} B ( {x}_{j} , t + {δ}_{i} ) \qquad ( 4 )$$

Equation (4) reverses the blending process of equation (1). It is important to point out that the extracted beam data will contain a lot of interferences from other overlapping beams. The interferences will appear random if one sorts the same data into domains other than the usual beam display. For example, one can first sort the extracted beam data into common receiver domain. In common receiver domain, interfering noises from other beams appear random by design. Signals belonging to corresponding beams are still coherent. The random noises in the common receiver domain can be attenuated using a simple method such as median filter [7] or a sophisticated algorithm such as singular value decomposition [8, 9], or any other similar methods. Another sorting is followed to reverse the first sorting, putting the data back into individual beams.

### 2.2 Implementation Considerations

Implementation of the BLEND data acquisition and processing requires some special cares as outlined below:
1. Time Gain Control (TGC) setting: TGC is used in commercial ultrasound scanners to boost reflections from deep tissues prior to analog to digital conversion. We need to reduce or remove TGC in BLEND data acquisition because a new beam is transmitted before deep reflections from previous beams are recorded. It is possible that we do not need TGC at all for the following reason: there are so much mixing of early (shallow) reflections with late (deep) reflections such that the resulting signal amplitudes are not varying dramatically as a function of lapse time.
2. Dither Time setting: we use (δᵢ=α*i+b*random ( ) in our design of dither time where the integer i is the beam sequence number, a is typically between 20-40 microseconds, b is typically set at 10 microseconds. The function random (is a random number generator whose value is in between −1 and +1.
3. Beam Sequence setting: To maximize the randomness in the mixing of a BLEND record we use another pseudo random sequence in selecting beams for a sequence of dither times. The selection criterion is to maximize overall spatial separation between beams.
4. Attenuation of Random Interferences after extraction of a beam: we do not need to be very aggressive in attenuating random interferences in extracted beam data. The reason is that the subsequent beamforming will automatically attenuate these random noises because they are not stationary and do not satisfy the imaging conditions implicit in all well-known beamforming algorithms.
5. Choice of Transducers: if there are two rows of elements in a transducer, we can dedicate one row for transmitting and another row for receiving. That is ideal for BLEND data acquisition. For conventional ultrasound transducers we can use odd elements for transmitting and even elements for receiving, or we can use all elements for both transmitting and receiving by allowing time gaps in the BLEND record when elements are used for transmission.
6. Choice of Beam Types: the design and process work for all beam types. We prefer focused beams or focusing beams because we can maximize spatial separation of beams.
7. Choice of Beamformer: we prefer advanced beamformer algorithm than conventional scanline-based dynamic focusing (in receivers). Double dynamic focusing in both transmitters and receivers has better ability in random noise attenuation.

### Part III: Example

### 3.1 Echo Data Simulation

We use a modified version of Fresnel Simulator from Ultrasound Toolbox (USTB, https://www.ustb.co) for generation of numerical ultrasound beam data. The use of this simulator is subject to the citation rule. We sincerely thank the authors for making it available in the public domain [11]. The simulator is based on Fresnel approximation of diffraction of acoustic waves for rectangular transducers in a linear time invariant (LTI) system. Inputs to the simulator include a phantom model specification, a transducer specification, and a waveform specification. The phantom model used in this simulation contains:
two rectangular boxes with a depth range between 7-9 mm,
4 flat continuous reflectors at 20 mm, 40 mm, 60 mm and 80 mm depth,
A hyperechoic target with 8 mm radius at 70 mm depth and a second hyperechoic target with 6 mm radius at 50 mm depth,
A row of scatter points at 30 mm depth and a column of scatter points at the center of the model.

FIG. 4 is a depict of the phantom model. The transducer is a linear array with 256 elements (0.2 mm in pitch size) and each element has a width of 0.18 mm and a height of 5 mm. The central frequency of the simulated echo data is 6 MHz with 80% useful bandwidth and sampling frequency was 48 MHz.

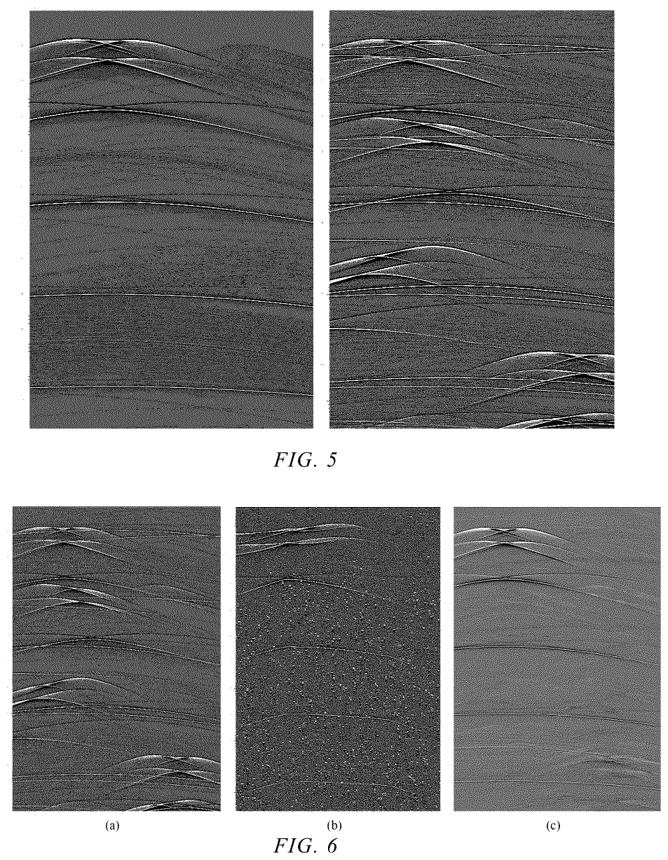

We have simulated a total of 256 focused beams using a Tx sub-aperture of 128 elements and a Rx sub-aperture of 256 elements. The focal depth is set at 25 mm. We add each beam into a BLEND buffer in a random order and with an increasing dither time. The dither time contains a random time perturbation. We store the dither times and beam coordinates for later de-blending use. FIG. 5 shows one focused beam near the center of the transducer (left) and a portion of the full BLEND record that is in accordance with the focused beam (right). The extracted BLEND records contain the same information as the focused beams, plus additional interference energies from neighboring beams. These interference energies need to be removed prior to beamforming.

### 3.2 Deblending Test

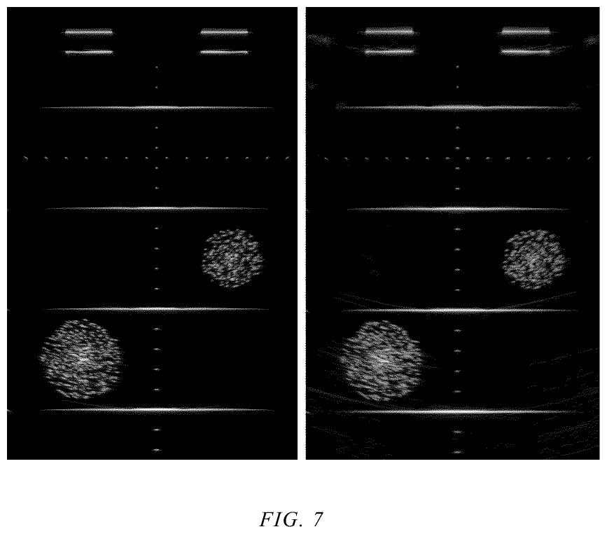

FIG. 6 illustrates the process and results of deblending. The left panel shows a blended beam record extracted from the full BLEND record. We first sort all blended beam records into common receiver gathers as shown in the middle panel. In the common receiver gather true acoustic signals are still coherent while interference energies appear random by design. We then apply a de-spike filter and a random noise removal filter to all common receiver gathers. Finally, we sort the data back into individual beam records. The panel on the right shows the same beam record after deblending. All interference energies from neighboring beams are effectively removed. True acoustic signals are almost intact.

### 3.3 Beamforming Test

FIG. 7 shows a comparison of an image of 256 focused beams acquired sequentially (left) and another image of the BLEND data that contains the same 256 focused beams (right). We see similar resolution and image quality between conventional ultrasound data acquisition and our BLEND data acquisition. The benefit of our BLEND data acquisition is in significant reduction of data acquisition time. The sequential data acquisition on the left takes 38.7 ms wall clock time. The BLEND data acquisition on the right takes 5 ms wall clock time.

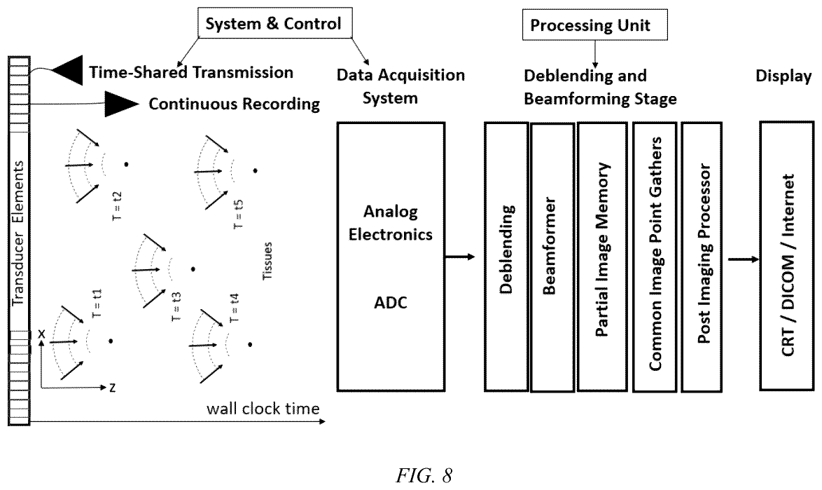

FIG. 8 is a schematic representation of the BLEND data acquisition and imaging architecture of one embodiment of the present invention. The system & control contains a data acquisition unit and one or more CPUs and one or more GPUs. One of the CPU or GPU sends instructions to the data acquisition system, at a certain time stamp, to transmit an acoustic pulse to each element of the transducer within a transmit aperture with a time delay that is specially designed for a given beam. After a short delay for transmission, the CPU or GPU sends instructions to the data acquisition system to switch each element of the transducer to receiving mode. The process is repeated until all beams are transmitted. The recording function in the data acquisition system is always on, continuously recording acoustic echoes reflected from tissue contrasts. The echo signals contain interferences from all beams since they are recorded into one long data buffer. They are sent to a special processing unit for deblending and beamforming on the CPUs, GPUs, or both. The final image is displayed on a local monitor or transmit via TCP/IP to a remote display device.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

### REFERENCES

[1] Richard S. C. Cobbold (2007), Foundations of Biomedical Ultrasound, Oxford University Press, pages 431-437.
[2]O. H. Schuck (1957), Variable Focus Transducer, U.S. Pat. No. 3,090,030, May 14, 1963.
[3]B. S. Hertzberg and W. D. Middleton (2016), Ultrasound: The Requisites, The Third Edition, Elsevier. Chapter 1, pages 3-31. Also at expertconsult.com.
[4]P. Suetens (2009), Fundamentals of Medical Imaging. 2nd Edition, Cambridge University Press, pages 33-158.
[5]G. Montaldo, M. Tanter, J. Bercoff, N. Benech, and M. Fink (2009), Coherent Planewave Compounding for very high Frame Rate Ultrasonography and Transient Elastography, IEEE Trans Ultrasonics, Ferroelectronics and Frequency Control, Vol. 56, pages 489-506.
[6]D. J. Napolitano, B. D. DeBusschere, G. W. McLaughlin, L. Y. Mo, C. H. Chou, T. L. Ji, R. W. Steins (2011), Continuous Transmit Focusing Method and Apparatus for Ultrasound Imaging Systems, U.S. Pat. No. 8,002,705, Issued August, 2011.
[7]M. A. Gungor and I. Karagoz (2016), The effects of the median filter with different window sizes for ultrasound image, 2nd IEEE International Conference on Computer and Communications (ICCC), 549-552, doi: 10.1109/CompComm.2016.7924761.
[8]C. Demené, T. Deffieux, M. Pernot, B F Osmanski, et. al. (2015), Spatiotemporal Clutter Filtering of Ultrafast Ultrasound Data Highly Increases Doppler and fUltrasound Sensitivity, IEEE Trans. Med. Imaging 34, 2271-2285.
[9]P. Song, J. D. Trzasko, A. Manduca, B. Qiang, R. Kadirvel, D. F. Kallmes, and S. Chen (2017), Accelerated Singular Value-Based Ultrasound Blood Flow Clutter Filtering With Randomized Singular Value Decomposition and Randomized Spatial Downsampling, IEEE Trans Ultrason Ferroelectr Freq Control. 64, 706-716. doi: 10.1109/TUFFC.2017.2665342.
[10]A. Rodriguez-Molares, Fresnel simulator, http://www.ustb.no/examples/fresnel/

What is claimed is:

1. A method of acquiring ultrasound radio-frequency data using a time-shared transmission and continuous recording (BLEND) design, comprising:
providing an ultrasound transducer, the ultrasound transducer including a plurality of elements;
providing an ultrasound data acquisition system, the ultrasound data acquisition system including analog electronics, an analog-to-digital converter, and a CPU (central processing unit) or GPU (graphic processing unit);
transmitting a plurality of ultrasound beams using a transmission function of the ultrasound transducer and the ultrasound data acquisition system;
receiving the ultrasound beams using a reception function of the ultrasound transducer and the ultrasound data acquisition system;
recording raw radio-frequency data with the ultrasound data acquisition system;
sending the raw radio-frequency data to a processing unit; and
deblending the raw radio-frequency data into individual ultrasound beam records,
wherein the ultrasound beams are transmitted in such a way that a subsequent ultrasound beam is transmitted before a previous ultrasound beam is received by the ultrasound transducer; and
wherein the ultrasound beams are overlapping in time in accordance with a first pseudo random sequence and overlapping in space in accordance with a second pseudo random sequence.

2. The method of claim 1, wherein the ultrasound transducer is a 1D linear array, 1D curved array, 1D phased array, or 2D matrix array.

3. The method of claim 1, wherein the elements are first used as emitters and subsequently used receivers.

4. The method of claim 1, wherein the elements include first row elements and second row elements; the first row elements are dedicated to transmission or reception; and the second row elements are dedicated to reception or transmission.

5. The method of claim 1, wherein the elements include odd elements and even elements; the odd elements are dedicated to transmission or reception; and the even elements are dedicated to reception or transmission.

6. The method of claim 1, wherein the data acquisition system includes analog electronics, one or more analog-to-digital converters, and one or more CPUs (or GPUs).

7. The method of claim 1, wherein a time gap (dither time) between two adjacent ultrasound beams is randomly chosen between 0 and 200 microseconds.

8. The method of claim 1, wherein deblending the ultrasound radio-frequency data comprises:
(i) taking the ultrasound radio-frequency data as an input;
(ii) extracting raw beam records by reversing dither times and applying receiver apodizations for the ultrasound beams;
(iii) sorting the raw beam records into a common receiver domain;
(iv) performing de-spike and random noise attenuation in the common receiver domain; and
(v) resorting the processed data back to obtain the individual ultrasound beam data.

9. A system for acquiring and processing ultrasound radio-frequency data using a time-shared transmission and continuous recording (BLEND) design, comprising:
an ultrasound transducer, the ultrasound transducer including a plurality of elements;
a data acquisition system, the data acquisition systems including analog electronics, an analog-to-digital converter, and a first CPU (central processing unit) or first GPU (graphic processing unit);
a display device;
a keyboard;
a pointing device;
a data acquisition device that includes analog to digital converters (ADC); and
a processing unit that includes a second CPUs or a second GPU,
wherein the first and second CPUs and the first and second GPUs are adapted to:
acquire, via the ultrasound transducer and the data acquisition system, raw radio-frequency data by transmitting a plurality of ultrasound beams in such a way that a subsequent ultrasound beam is transmitted before a previous ultrasound beam is received by the ultrasound transducer;
record the raw radio-frequency data with the data acquisition system;
send the raw radio-frequency data to the processing unit;
deblend the raw radio-frequency data into individual ultrasound beam data;
process and send the deblended ultrasound beam data to CPU memories or GPU memories;
beamform the deblended ultrasound beam data on the first and second CPUs or the first and second GPUs to obtain an ultrasound image; and
process and send the ultrasound image to the display device.

10. The system of claim 9, wherein the display device is connected to the processing unit remotely, via internet connection, wireless connection, or satellite connection.

11. The system of claim 9, wherein the ultrasound transducer is a 1D linear array, 1D curved array, 1D phased array, or 2D matrix array.

12. The system of claim 9, wherein the keyboard is a wireless keyboard or a software keyboard installed on the processing unit.

13. The system of claim 9, wherein the elements are first used as emitters and subsequently used receivers.

14. The system of claim 9, wherein the elements include first row elements and second row elements; the first row elements are dedicated to transmission or reception; and the second row elements are dedicated to reception or transmission.

15. The system of claim 9, wherein the elements include odd elements and even elements; the odd elements are dedicated to transmission or reception; and the even elements are dedicated to reception or transmission.

16. The system of claim 9, wherein deblending the raw radio-frequency data comprises:
(i) taking the ultrasound radio-frequency data as an input;
(ii) extracting raw beam records by reversing dither times and applying receiver apodizations for the ultrasound beams;
(iii) sorting the raw beam records into a common receiver domain;
(iv) performing de-spike and random noise attenuation in the common receiver domain; and
(v) resorting the processed data back to obtain the individual ultrasound beam data.

17. The system of claim 9, wherein the data acquisition system and the processing unit share a same CPU or a same GPU, and the first CPU is the same as the second CPU or the first GPU is the same as the second GPU.
